(19) United States
(12) Patent Application Publication
(10) Pub. No.: US 2010/0041587 A1
(43) Pub. Date: Feb. 18, 2010

(54) COMBINATION THERAPY FOR INHIBITION OF PLATELET AGGREGATION

(76) Inventors: R. Stephen PORTER, Franklin, TN (US); Kristen K. Flaharty, Fort Myers, FL (US); James E. Tcheng, Durham, NC (US); John W. Ferkany, Brighton, MA (US)

Correspondence Address: CLARK & ELBING LLP, 101 FEDERAL STREET, BOSTON, MA, 02110, US

(21) Appl. No.: 12/540,062

(22) Filed: Aug. 12, 2009

Related U.S. Application Data
(63) Continuation of application No. 11/515,596, filed on Sep. 5, 2006.
Application No. 11/515,596 is a continuation of application No. PCT/US05/07440, filed on Mar. 7, 2005.
(60) Provisional application No. 60/550,792, filed on Mar. 5, 2004.

(51) Int. Cl. A61K 38/16; A61K 31/727

(52) U.S. Cl. 514/8; 514/56

(57) ABSTRACT

The invention features methods for preventing platelet activation and aggregation and for treating individuals suffering from conditions or undergoing procedures that may result in unwanted platelet aggregation. The methods are based on the intravenous, subcutaneous, or transdermal administration of a platelet activation or aggregation inhibitor, e.g., xemilofiban, followed by oral administration of the same or a different platelet activation or aggregation inhibitor. The treatment may commence prior to a medical or surgical procedure or after the outbreak of an adverse medical condition, either of which results in the activation of platelets that may lead to thrombus formation, and may continue thereafter.

### CROSS-REFERENCE TO RELATED APPLICATIONS

This application is a continuation of U.S. patent application Ser. No. 11/515,596, filed Sep. 5, 2006, which is a continuation of International Application No. PCT/US2005/007440, filed Mar. 7, 2005, which, in turn, claims benefit of U.S. Provisional Patent Application Serial No. 60/550,792, filed Mar. 5, 2004, the disclosures of which are each hereby incorporated by reference in their entirety.

### BACKGROUND OF THE INVENTION

The invention relates to the field of medical treatments, in particular the inhibition of platelet aggregation.

Fibrinogen is a glycoprotein present as a normal component of blood plasma. It participates in platelet aggregation and fibrin formation in the blood clotting mechanism. Platelets are cellular elements found in whole blood, which also participate in blood coagulation. Fibrinogen binding to platelets is important for normal platelet function in the blood coagulation mechanism. When a blood vessel receives an injury, the platelets binding to fibrinogen will initiate aggregation and form a thrombus. Injury can occur during medical or surgical procedures. In addition, certain medical conditions, such as diabetes, leads to platelet aggregation and thrombosis in vital organs. Interaction of fibrinogen with platelets occurs through a membrane glycoprotein complex, known as glycoprotein IIb/IIIa (GPIIb/IIIa). Inhibitors of this interaction are useful in modulating or preventing platelet thrombus formation.

The activation of platelets and the resultant aggregation have been shown to be important factors in the pathogenesis of acute coronary syndrome, unstable angina pectoris, transient myocardial ischemia, acute myocardial infarction, peripheral arterial occlusion, and atherosclerosis. In most of these serious cardiovascular disorders, intracoronary or intra-arterial thrombus is present. The thrombus is generally formed by activated platelets that adhere and aggregate at the site of endothelial injury or plaque rupture. Because of the relative contribution of activated platelets to aggregation and subsequent formation of an occlusive thrombus, antiplatelet agents have been developed that inhibit platelet activation or aggregation.

### SUMMARY OF THE INVENTION

The present invention provides methods for preventing platelet activation and aggregation and for treating individuals suffering from conditions or undergoing procedures that may result in unwanted platelet aggregation. The methods are based on the intravenous, subcutaneous, or transdermal administration of a platelet activation or aggregation inhibitor, e.g., xemilofiban, followed by oral administration of the same or a different platelet activation or aggregation inhibitor. The treatment may commence prior to a medical or surgical procedure or after the outbreak of an adverse medical condition, either of which results in the activation of platelets that may lead to thrombus formation, and may continue thereafter.

In one aspect, the invention features a method of inhibiting platelet aggregation in a subject including the steps of intravenously, subcutaneously, or transdermally (e.g., via a patch, sonophoresis, a microneedle array, or iontophoresis) administering a first platelet activation or aggregation inhibitor to the subject; and orally administering a second platelet activation or aggregation inhibitor to the subject, provided that, when the first platelet activation or aggregation inhibitor is heparin, the second platelet activation or aggregation inhibitor is not aspirin, and provided that when the first platelet activation or aggregation inhibitor is RPR 109891, the second platelet activation or aggregation inhibitor is not RPR 109891. The first and second platelet activation or aggregation inhibitors may be the same or different. The method may further include administering a cholesterol lowering agent, an agent that modifies eicosanoid activity, a 5HT2a antagonist, nonsteroidal anti-inflammatory drugs, an adrenergic inhibitor, an angiotensin converting enzyme inhibitor, an angiotensin II receptor antagonist, a fibrilyinic agent, a beta blocker, a calcium channel blocker, a diuretic agent, a steroid, a steroidal glycoside, a nicotinic acid drug, a bile acid sequestrant, a fibrate, ETC 588 (liposome), ETC 216 (ApoA-I Milano/phospholipid complex), ETC 642 (RLT Peptide), pirozadil, or a vasodilator or to the subject, e.g., with the first or second platelet aggregation inhibitor or separately.

Exemplary first or second platelet activation or aggregation inhibitors include a GP IIb/IIIa antagonist, a heparin, tissue plasminogen activator, a Factor Xa inhibitor, a purinergic-receptor antagonist, a thrombin inhibitor, a phosphodiesterase inhibitor (e.g., dipyridamole), a cyclooxygenase inhibitor (e.g., aspirin), a CD40 antagonist, and a leukotriene inhibitor. Examples of GP IIb/IIIa antagonists include tirofiban, abciximab, eptifibatide, TRM-147, SM-20302, L-378167, rClf A, ME-3230, SR-121787, UR-12947, L-734217, DMP-757, EMD-96717, SDZ-GPI-562, RG-13965, SB-207448, SC-56929, RWJ-50042, UR 4005, L-703014, SKF-106760, CRL-42796, HMR-1794, CGH-400, Ro-43-5054, Barbourin, Bitistatin, SC-49992, TP-9201, MA-16N7C2, roxifiban (DMP-754), lamifiban, xemilofiban, lotrafiban, sibrafiban, DU-728, DMP-728, MK-852, SC-52012A, echistatin, TAK-029, ME-3277, T-250, MS-180, TA-993, elarofiban (RWJ-53308), cromafiban (CT-50352), YM-337, lefradafiban (BIBU-104), fradafiban (BIBU-52), ZD-2486, RPR-109891, gantofiban, GR-144053F, and pharmaceutically acceptable salts thereof. Exemplary heparins include low molecular weight heparins, such as ardeparin, certoparin, dalteparin, enoxaparin, nadroparin, reviparin, SNAD-UFH, SNAC-UFH, or tinazaparin. Factor Xa inhibitors include coumadin, danaparoid, fondaparinux, CL-1031, DPC 906, Sanorg-34006, MCM 16, MCM 17, BAY 59-7939, KFA-1982, GH9001, DPC423, ZD4927, DX-9065a, YM 60828, SR 90107, FXV673, and tifacogin. The purinergic-receptor antagonist is for example a P2Y₁₂ or P2Y₁ antagonist or both. P2Y₁₂ antagonists include clopidogrel, cangrelor, and ticlopidine, and P2Y₁ antagonists include ATP and MRS 2179 (2′-deoxy-N-6-methyladenosine 3′,5′-bisphosphate). Exemplary thrombin inhibitors re bivalirudin, lepirudin, argatroban, melagatran, ximelagatran, antithrombin 1111, dermatan, mesoglycan, MB-015, H-376/95, BIBR 1048, efegatran, TRI-50B, inogatran, V19, and PEG-r-hirudin. CD40 antagonists include soluble CD40 ligand and a CD40 antibody, and leukotriene inhibitors include monelukast, zafirlukast, and zileuton.

When the subject has a body mass index of greater than 30, 4 to 10 mg of the first platelet activation or aggregation inhibitor is typically administered, and when the subject has a body mass index of less than 30, 1 to 3 mg of the first platelet activation or aggregation inhibitor is typically administered.

The intravenous, transdermal, or subcutaneous administration may also occur prior to or during a medical or surgical procedure, e.g., a cardiovascular interventional procedure. Exemplary cardiovascular interventional procedures include percutaneous transluminal coronary angioplasty (PTCA), percutaneous coronary intervention (PCI), coronary artery stent procedure, cardiac bypass surgery (CABG), peripheral transluminal angioplasty (PTA), peripheral vascular stent implantation, and an angioplasty procedure, such as atherectomy, balloon angioplasty, laser angioplasty, intracranial angioplasty, or angioplasty of peripheral arteries. The cardiovascular interventional procedure may be performed on a coronary, carotid, iliac, renal, popliteal, superficial femoral, or femoral artery or any other suitable artery or vein. The cardiovascular interventional procedure may or may not include implanting a stent, e.g., a drug eluting stent, or the insertion of a coronary catheter into the subject. Prior to the administration of the first platelet activation or aggregation inhibitor, the subject may be diagnosed as suffering from an acute myocardial infarction. When the subject is diabetic, the second platelet activation or aggregation inhibitor is typically administered for 3 to 30 days, e.g., 7 to 14 days. Administration of the first platelet activation or aggregation inhibitor may also occur prior to or during transportation to or from a medical facility. The administration of the second platelet activation or aggregation inhibitor may occur after a surgical or medical procedure.

In certain embodiments, the first platelet activation or aggregation inhibitor is a first GP IIb/IIIa antagonist, and the second platelet activation or aggregation inhibitors is a second GP IIb/IIIa antagonist. The first platelet activation or aggregation inhibitor may be administered as a loading dose or a continuous infusion. For a loading dose, the first platelet activation or aggregation inhibitor is, for example, tirofiban, abciximab, xemilofiban, or eptifibatide. In addition, when employing a loading dose, the second GP IIb/IIIa antagonist may be administered for at least 2 days, e.g., at least 7, 14, or 30 days. In these embodiments, the second platelet activation or aggregation inhibitor is, for example, xemilofiban. The loading dose may deliver 0.3 to 60 mg, e.g., 1 to 10 mg or 3 to 6 mg, of the first platelet activation or aggregation inhibitor. When employing a continuous infusion, the first platelet activation or aggregation inhibitor is administered, for example, for at least 6, 12, 18, 24, or 48 hours. The continuous infusion may deliver the first platelet activation or aggregation inhibitor at a rate of 0.01 mg/kg/min to 1 mg/kg/min, e.g., about 0.135 mg/kg/min. The continuous infusion may deliver 0.3 to 60 mg, e.g., 1 to 10 mg or 3 to 6 mg, of the first platelet activation or aggregation inhibitor. In one embodiment, the continuous infusion administers 3 to 6 mg of xemilofiban. In another embodiment, an infusion of tirofiban is administered for at least 18, 24, or 48 hours; an infusion of abciximab is administered for at least 12, 18, 24, or 48 hours; an infusion of xemilofiban is administered for at least 18, 24, or 48 hours; or an infusion of eptifibatide is administered for at least 18, 24, or 48 hours. Following a continuous infusion, the second platelet activation or aggregation inhibitor, e.g., xemilofiban, may be administered for at least 1 day, e.g., at least 2, 7, 14, or 30 days.

The invention further features a method of inhibiting platelet aggregation in a subject including subcutaneously administering a platelet aggregation, a Factor Xa inhibitor, a heparin, and a thrombin inhibitor to the subject. The platelet aggregation inhibitor is, for example, a GP IIb/IIIa antagonist, a purinergic-receptor antagonist, a phosphodiesterase inhibitor, or a cyclooxygenase inhibitor, as described herein. Exemplary Factor Xa inhibitors, heparins, and thrombin inhibitors are also described herein.

In another aspect, the invention features a kit containing a combination of therapeutic agents as described in the methods of the invention. For example, one kit includes a first platelet activation or aggregation inhibitor formulated for intravenous, transdermal, or subcutaneous administration; and a second platelet activation or aggregation inhibitor formulated for oral administration, wherein said first and second platelet activation or aggregation inhibitors are the same or different. Another kit includes a platelet aggregation inhibitor formulated for subcutaneous administration, a Factor Xa inhibitor, a heparin, and a thrombin inhibitor. Kits may further include instructions for administering the therapeutic compounds as described herein. Additional compounds may be included in a kit of the invention as disclosed.

The therapeutic compounds of the invention may be administered as pharmaceutically acceptable salts, prodrugs, or derivatives.

By “cardiovascular interventional procedure” is meant a medical or surgical procedure that repairs or prevents damage to the heart and associated arteries and veins. Exemplary arteries on which a cardiovascular interventional procedure are performed include the coronary, carotid, iliac, renal, popliteal, superficial femoral, and femoral arteries. A procedure may also be performed on any other suitable artery or vein.

By “pharmaceutically acceptable salt” is meant a non-toxic salt of a compound of the invention formed, e.g., from non-toxic inorganic or organic acids. Such non-toxic salts include, for example, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Other pharmaceutically acceptable salts are known to those skilled in the art.

By “platelet activation or aggregation inhibitor” is meant a compound that reduces the ability of platelets to activate or aggregate in vivo.

By “prodrug” is meant a compound which is rapidly transformed in vivo to a therapeutic agent, for example, by hydrolysis in blood (T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins, et al. Synthetic Communications, 26(23), 4351-4367 (1996)).

By “therapeutically effective amount” is meant an amount of a pharmaceutical composition sufficient to produce a preventative, healing, curative, stabilizing, or ameliorative effect either in the treatment of a disorder or in the treatment of symptoms of a disorder.

By “treating” is meant the medical management of a patient with the intent that a prevention, cure, stabilization, or amelioration of the symptoms will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of the disorder; and supportive treatment, that is, treatment employed to supplement another specific therapy directed toward the improvement of the disorder. The term “treatment” also includes symptomatic treatment, that is, treatment directed toward constitutional symptoms of the disorder.

By “unwanted platelet aggregation” is meant the aggregation of platelets, e.g., in blood vessels, in a patient that causes a detrimental effect to the patient.

The methods of the invention are advantageous because they reduce the cost of initial dosing by limiting the amount of therapeutic agent administered. In addition, oral dosing reduces the cost of continued dosing and does not require inpatient care, allowing therapy to be continued in an outpatient setting. Transdermal, subcutaneous, and oral administration carry a reduced risk of infection compared to intravenous due to the absence of indwelling catheters. Oral and transdermal care also increase patient comfort because of a lack of repeated injections. Transdermal delivery also allows tightly controlled titration of dose and continuous drug delivery. Subcutaneous administration will allow for longer duration of absorption of a therapeutic agent and thus duration of therapy.

Other features and advantages of the invention will be apparent from the following description and claims.

### BRIEF DESCRIPTION OF THE DRAWING

FIG. 1 is a table of structures of selected compounds described herein.

### DETAILED DESCRIPTION OF THE INVENTION

Certain medical and surgical procedures and medical conditions may cause unwanted platelet aggregation in individuals. Accordingly, the invention features a method for inhibiting platelet aggregation in patients at risk thereof. Exemplary platelet activation or aggregation inhibitors are described herein.

In one embodiment, the methods of the invention include the intravenous, subcutaneous, or transdermal administration of a platelet activation or aggregation inhibitor followed by the oral administration of the same or a different platelet activation or aggregation inhibitor. As described herein, the method may be used in conjunction with a medical or surgical procedure or in treatment for an adverse medical condition.

Another method of inhibiting platelet aggregation includes subcutaneously administering a platelet activation or aggregation inhibitor; administering a Factor Xa inhibitor; administering a heparin; and administering a thrombin inhibitor to a subject.

Platelet stimulation and subsequent aggregation are known to cause the expression or release of several factors that could affect vascular pathology. These include TXA2, a co-stimulator of platelets that has vasoconstrictive activity; P-selectin, an α granule protein that mediates platelet rolling, leukocyte adhesion, and coagulation; ADP and serotonin, which amplify platelet aggregation; platelet-derived growth factor, a growth factor for vascular cells; and CD40L, a member of the tumor necrosis factor family of proteins (reviewed in Platelets in Thrombotic and Non-thrombotic Disorders. New York, N.Y.: Cambridge University Press; 1992.). Although any of these factors could contribute to long-term vascular pathologies, CD40L appears to be particularly relevant because this protein is now known to be prothrombotic (Nat. Med. 2002; 8:247-252) and proinflammatory (Proc Natl Acad Sci USA. 2000; 97:7458-7463), to have a proven role in atherosclerotic lesion progression (Circulation. 2001; 104:2266-2268), and to be a risk factor for cardiovascular events. The majority of CD40L is found in platelets and thus released under conditions of activation and aggregation.

### Pharmaceutical Agents

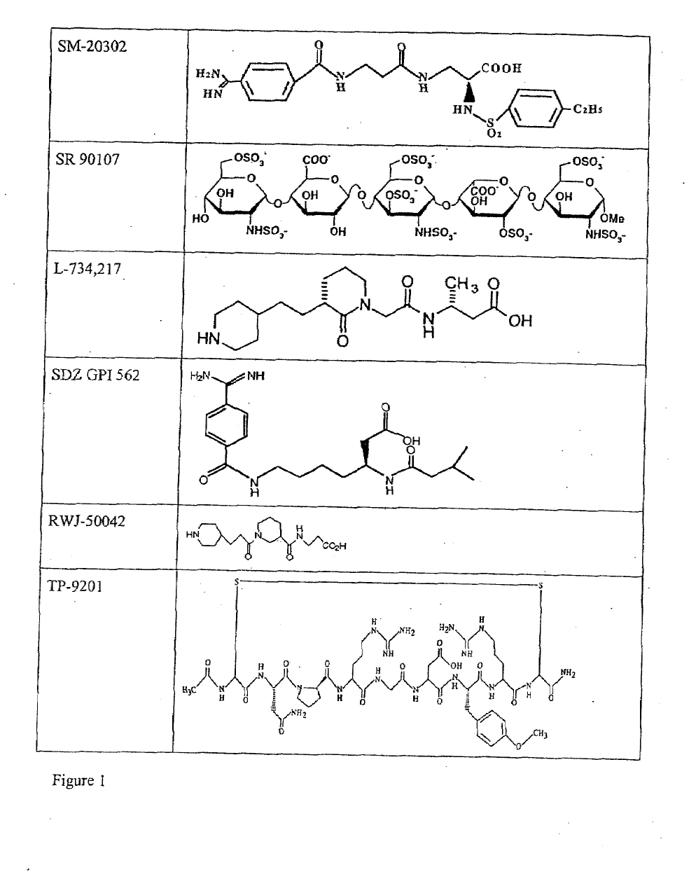

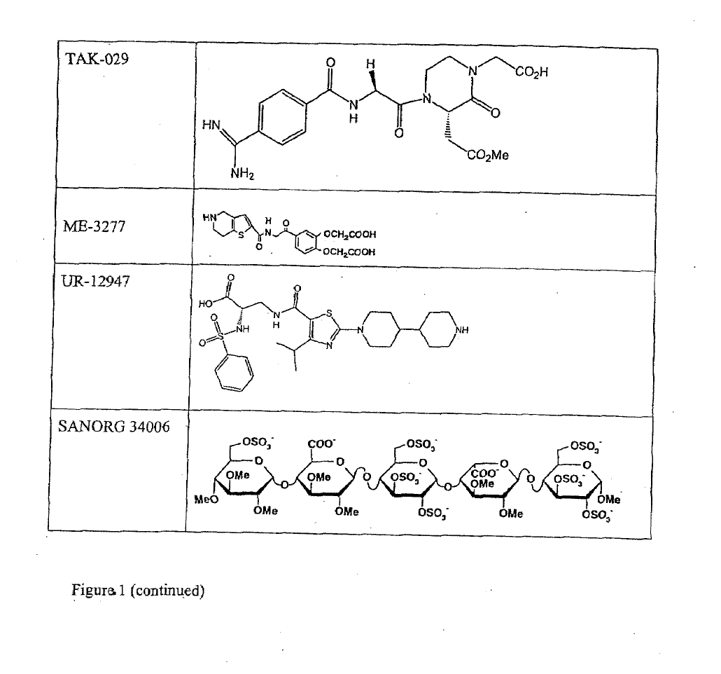

The treatment methods of the invention may employ a variety of therapeutic agents. Exemplary platelet activation or aggregation inhibitors include glycoprotein IIb/IIIa antagonists, heparins, tissue plasminogen activator, Factor Xa inhibitors, purinergic-receptor antagonists, thrombin inhibitors, phosphodiesterase inhibitors (e.g., dipyridamole), cyclooxygenase inhibitors (e.g., aspirin), CD40 antagonists, and leukotriene inhibitors. Selected structures are shown in FIG. 1

Antagonists for the glycoprotein IIb/IIIa receptor are known in the art. Such antagonists include, without limitation, tirofiban, abciximab, eptifibatide, TRM-147, SM-20302, L-378167, rClf A, ME-3230, SR-121787 (ethyl 3-[N-[4-[4-[amino[(ethoxycarbonyl) imino]methyl]phenyl]-1,3-thiazol-2-yl]-N-[1-[(ethoxycarbonyl)methyl]piperid-4-yl]amino]propionate), UR-12947, L-734217, DMP-757, EMD-96717, SDZ-GPI-562, RG-13965, SB-207448 (8-[[[4-(aminoiminomethyl)phenyl]amino]-carbonyl]-2,3,4,5-tetrahydro-3-oxo-4-(2-phenylethyl)-1H-1,4-benzodiazepine-2-acetic acid dihydrochloride), SC-56929, RWJ-50042, UR 4005, L-703014, SKF-106760 (cyclo(S,S)—N^{α}-2-mercaptobenzoyl-N^{α}-methylarginyl-glycyl-aspartyl-2-mercaptophenyl-amide), CRL-42796 ((2S)-2-[(2-naphthylsulfonyl)amino]-3-{[2-({4-(4-piperidinyl)-2-[2-(4-piperidinyl)ethyl]butanoyl}amino)acetyl]amino}propanoic acid dihydrochloride), HMR-1794, CGH-400, Ro-43-5054 (N—(N—[N-(p-amidinobenzoyl)-beta-alanyl]-L-alpha-aspartyl)-3-phenyl-L-alanine-trifluoracetate), Barbourin, Bitistatin, SC-49992 (8-guanidino-octanoyl-aspartic acid-phenylalanine), TP-9201, MA-16N7C2, roxifiban (DMP-754), lamifiban, xemilofiban, lotrafiban, DU-728, DMP 728 ([cyclo (D-2-aminobutyrate-N-methyl-L-arginyl-glycyl-L-asparty]) 3-aminomethyl-benzoic acid]), MK-852 (cyclic disulfide N-acetyl-cys-asn-(5,5-dimethyl-4-thiazolidine-carbonyl)-(4-aminomethyl-phe)-gly-asp-cys, monoacetate (all L-amino acids)), SC-52012A, echistatin, TAK-029, ME-3277, T-250, MS-180 ((S)-(−)-ethyl[6-[4-(morpholinoformimidoyl)benzamido]-3,4-dihydro-2H-1-benzo-pyran-3-yl]acetate hydrochloride), TA-993 ((−)-cis-3-acetoxy-5-(2-(dimethylamino)ethyl)-2,3-di-hydro-8-methyl-2-(4-methylphenyl)-1,5-benzothiazepin-4(5H)one maleate), elarofiban (RWJ-53308), cromafiban (CT-50352), sibrafiban, YM-337, lefradafiban (BIBU-104), fradafiban (BIBU-52), ZD-2486, RPR-109891, UR-3216 (Cardiovascular Drug Reviews 2001, 19: 2540), gantofiban, GR-144053F (((4-[4-[4-(aminoiminomethyl]-1-piperazinyl]-1-piperidineacetic acid, hydrochloride trihydrate)), and pharmaceutically acceptable salts thereof. Additional glycoprotein IIb/IIIa receptor antagonists are disclosed in U.S. Pat. Nos. 5,470,849; 5,463,011; 5,455,243; 5,451,578; 5,446,056; 5,441,952; 5,422,249; 5,416,099; 5,405,854; 5,397,791; 5,393,760; 5,389,631; 5,380,713; 5,374,622; 5,358,956; 5,344,783; 5,340,798; 5,338,723; 5,334,596; 5,321,034; 5,318,899 (e.g. cyclic heptapeptides Mpr-(Acetimidyl-Lys)-Gly-Asp-Trp-Phe-Cys-NH₂, Mpr-(Acetimidyl-Lys)-Gly-Asp-Trp-Phe-Pen-NH₂, Mpr (Phenylimidyl-Lys)-Gly-Asp-Trp-Phe-Pen-NH₂, and Mpr-(Phenylimidyl-Lys)-Gly-Asp-Trp-Phe-Cys-NH₂, wherein Mpr is mercapto propionyl); U.S. Pat. Nos. 5,312,923; 5,294,616; 5,292,756; 5,281,585; 5,272,158; 5,264,420; 5,260,307; 5,239,113 (e.g. Ethyl 3S-[[4-[[4-(aminoiminomethyl)phenyl]amino]-1,4-dioxobutyl]amino]-4-pentynoate (xemilofiban)), U.S. Pat. Nos. 5,227,490; 5,206,373; and 4,703,036 (e.g. N-Methyl-D-phenylalanyl-N-[(1S)-1-formyl-4-guanidinobutyl]-L-prolinamide); and EP 505 868 (e.g. ((1-(2-((4-(aminoiminomethyl)benzoyl)amino)-3-(4-hydroxyphenyl)-1-oxopropyl)-4-piperidinyl)oxy)-(S)-acetic acid); WO 9311152 (e.g. N-(2-(2-(((3-((aminoiminomethyl)amino)propyl)amino)carbonyl)-1-piperidinyl)-1-(cyclohexylmethyl)-2-oxoethyl)-(R,S)glycine); EP 333 356; and WO 9422820.

Heparins may be fractionated, e.g., low molecular weight heparins, or unfractionated. Examples of low molecule weight beparins are ardeparin, certoparin, dalteparin, enoxaparin, nadroparin, reviparin, SNAD-UFH, SNAC-UFH, and tinazaparin.

Exemplary factor Xa inhibitors include coumadin, danaparoid, fondaparinux, CL-1031, DPC 906 (razaxaban), Sanorg-34006, MCM 16, MCM 17, BAY 59-7939, KFA-1982, GH9001, DPC423 (1-[3-(aminomethyl)phenyl]-N-[3-fluoro-2′-(methylsulfonyl)[1,1′-biphenyl]-4-yl]-3-(trifluoromethyl)-1H-pyrazole-5-carboxamide), ZD4927, DX-9065a (2S)-{4-[1-acetimidoyl-(3S)-pyrrolidinyl]oxyphenyl}-3-(7-amidino-2-naphthyl)propionic acid), YM 60828 (([N-[4-[(1-acetimidoyl-4-piperidyl)oxy]phenyl]-N-[(7-amidino-2-naphthyl)methyl]sulfamoyl]acetic acid dihydrochloride)), SR 90107, FXV673 (methyl-3-(4′-n-oxopyridylphenoyl)-3-methyl-2-(m-amidinobenzyl)-propionate), and tifacogin. Additional Factor Xa ihibitors are disclosed in U.S. Pat. No. 6,677,452, US20040006099A1, US20040006062A1, U.S. Pat. No. 6,673,810, U.S. Pat. No. 6,667,332, US20030232804A1, US20030232789A1, U.S. Pat. No. 6,664,393, WO03099276A1, US20030225144A1, EP1367054A1, EP1366045A2, US20030212117A1, US20030212054A1, U.S. Pat. No. 6,645,992, US20030207882A1, EP1358909A1, US20030199689A1, U.S. Pat. No. 6,635,657, WO03084929A1, WO03084533A1, US20030191115A1, EP1349847A1, U.S. Pat. No. 6,630,505, U.S. Pat. No. 6,630,468, U.S. Pat. No. 6,630,451, US20030181466A1, EP1345900A1, EP1337251A1, U.S. Pat. No. 6,610,701, US20030158120A1, U.S. Pat. No. 6,602,871, US20030144287A1, U.S. Pat. No. 6,599,926, EP1326838A1, WO03053925A1, U.S. Pat. No. 6,586,459, U.S. Pat. No. 6,586,418, EP1025086B1, EP0906094B1, U.S. Pat. No. 6,583,173, WO03050109A1, WO03049737A1, WO03049735A1, WO03049681A3, WO03049681A2, WO03048158A1, WO03048081A3, WO03048081A2, WO03047520A2, US20030109517A1, US20030105105A1, WO03044014A1, WO03043981A1, EP1314733A1, EP1175419B1, U.S. Pat. No. 6,569,874, US20030096820A1, U.S. Pat. No. 6,562,828, US20030078255A1, U.S. Pat. No. 6,548,525, U.S. Pat. No. 6,548,512, US20030069258A1, US20030069237A1, EP0946528B1, WO03026652A1, US20030065176A1, EP1296982A1, EP1296977A1, U.S. Pat. No. 6,541,639, U.S. Pat. No. 6,541,488, EP1294684A2, EP1294394A1, U.S. Pat. No. 6,538,017, U.S. Pat. No. 6,534,629, US20030050315A1, US20030049704A1, U.S. Pat. No. 6,525,076, WO03013531A1, U.S. Pat. No. 6,521,614, WO03010160A3, WO03010160A2, U.S. Pat. No. 6,515,142, WO03007942A1, US20030022916A1, US20030021773A1, US20030018023A1, U.S. Pat. No. 6,509,335, U.S. Pat. No. 6,506,771, EP1272458A1, U.S. Pat. No. 6,504,031, US20030004344A1, US20030004167A1, U.S. Pat. No. 6,500,855, U.S. Pat. No. 6,500,851, U.S. Pat. No. 6,500,803, WO02102380A1, WO02100886A1, WO02100847A3, WO02100830A1, U.S. Pat. No. 6,495,562, U.S. Pat. No. 6,492,368, US20020183324A1, US20020177713A1, U.S. Pat. No. 6,486,154, US20020173656A1, EP0892780B1, EP1254136A1, WO02085356A1, WO02085353A1, EP1070714A4, U.S. Pat. No. 6,472,562, U.S. Pat. No. 6,469,039, U.S. Pat. No. 6,469,029, U.S. Pat. No. 6,465,656, U.S. Pat. No. 6,458,793, U.S. Pat. No. 6,458,789, EP1242366A1, US20020119986A1, US20020115854A1, U.S. Pat. No. 6,436,985, EP1230382A2, U.S. Pat. No. 6,429,205, US20020103202A1, EP1226123A1, U.S. Pat. No. 6,426,346, US20020099225A1, WO02051831A1, U.S. Pat. No. 6,413,980, U.S. Pat. No. 6,410,733, U.S. Pat. No. 6,410,536, U.S. Pat. No. 6,407,256, WO0246159A1, EP1051176A4, U.S. Pat. No. 6,403,583, U.S. Pat. No. 6,399,644, U.S. Pat. No. 6,395,731, US20020061917A1, US20020055641A1, WO0230880A3, WO0230880A2, EP1196412A1, U.S. Pat. No. 6,372,759, WO0228836A1, U.S. Pat. No. 6,369,227, U.S. Pat. No. 6,369,080, U.S. Pat. No. 6,369,063, U.S. Pat. No. 6,365,617, WO0224690A3, WO0224690A2, WO0224654A1, WO0216315A1, US20020025963A1, US20020022614A1, EP0693925B1, US20020016326A1, WO0208199A3, WO0208199A2, EP1175419A2, U.S. Pat. No. 6,342,609, U.S. Pat. No. 6,339,099, WO0202519A3, WO0202519A2, U.S. Pat. No. 6,337,405, U.S. Pat. No. 6,337,344, WO0200655A1, WO0200651A3, WO0200651A2, WO0200647A1, EP0994893B1, U.S. Pat. No. 6,333,321, WO0195931A1, EP1161279A1, U.S. Pat. No. 6,329,527, WO0192251A3, WO0192251A2, EP1157010A1, U.S. Pat. No. 6,313,151, EP1140941A1, EP1140901A2, EP1140871A1, EP1140862A1, U.S. Pat. No. 6,287,794, US20010020033A1, U.S. Pat. No. 6,284,796, WO0158859A1, WO0155146A1, WO0151484A1, U.S. Pat. No. 6,262,047, U.S. Pat. No. 6,258,822, WO0144172A1, U.S. Pat. No. 6,248,770, U.S. Pat. No. 6,245,743, WO0133217A3, WO0133217A2, WO0132628A1, U.S. Pat. No. 6,228,854, WO0129006A1, U.S. Pat. No. 6,218,382, US20010000179A1, U.S. Pat. No. 6,211,341, U.S. Pat. No. 6,211,183, WO019795A1, U.S. Pat. No. 6,204,268, U.S. Pat. No. 6,197,748, U.S. Pat. No. 6,194,435, U.S. Pat. No. 6,191,159, U.S. Pat. No. 6,187,797, WO0105784A1, EP1070714A1, EP1064270A2, WO0078749A1, U.S. Pat. No. 6,165,974, EP1058549A1, U.S. Pat. No. 6,150,379, EP1051176A1, U.S. Pat. No. 6,133,256, WO0059902A3, WO0059902A2, and EP1042299A1, each of which is hereby incorporated by reference.

Purinergic-receptor antagonists are, for example, P2Y₁₂ antagonists or P2Y₁ antagonists. P2Y₁₂ antagonists include clopidogrel, cangrelor, and ticlopidine, and P2Y₁ antagonists include ATP and MRS 2179. Additional P2Y₁₂ inhibitors are disclosed in WO0216381A3, WO0216381A2, WO0146454A1, EP1240350A1, US20020052337A1, EP1240350A4, and US20030170777A1, each of which is hereby incorporated by reference. Additional P2Y₁ receptor antagonists are disclosed in WO0214511A3, WO0214511A2, EP1311677A2, U.S. Pat. No. 6,617,439, U.S. Pat. No. 6,476,211, U.S. Pat. No. 6,447,771, U.S. Pat. No. 6,387,645, U.S. Pat. No. 6,350,447, U.S. Pat. No. 6,335,013, EP0929218A4, EP0929218A2, WO9818430A2, WO9803178A2, J Med Chem. 2003; 46: 4974-87; J Med Chem. 2002; 45: 2090-100, J Med Chem. 2000; 43: 829-42, J Med Chem. 2002; 45: 208-18, Br J Pharmacol. 2002; 135: 2004-10, J Med Chem. 1999; 42: 1625-38, J Med Chem. 1998; 41: 183-90, J Med Chem. 2001; 44: 3092-108, J Med Chem. 2000; 43: 2196-203, J Med Chem. 2002; 45: 5694-709, Br J Pharmacol. 2002; 135: 1839-40, J Med Chem. 2000; 43: 746-55, J Med Chem. 2001; 44: 340-9, Mol Pharmacol. 1996; 50: 1323-9, J Med Chem. 2002; 45: 962-72, Br J Pharmacol. 1998; 124: 1-3, Nucleosides Nucleotides Nucleic Acids. 2001; 20: 333-41, Farmaco. 2001 January-February; 56: 71-5, Biochem Biophys Res Commun. 2000 Jun. 7; 272: 327-31, J Org Chem. 2002; 67: 8063-71, Neurosci Lett. 2000; 284: 179-81, J Med Chem. 1998; 41: 1456-66, Eur J Pharmacol. 2001; 412: 213-21, J Med Chem. 1999; 42: 5338-47, Cardiovasc Drug Rev. 2003; 21: 67-76, J Med Chem. 2001; 44: 749-62, FEBS Lett. 2003; 536: 145-50, Naunyn Schmiedebergs Arch Pharmacol. 2000; 362: 310-23, Br J Pharmacol. 2002; 135: 537-45, Curr Pharm Des. 2002; 8: 2371-99, Br J Pharmacol. 2000; 129: 1506-12, Platelets. 2002; 13: 285-92, J Med Chem. 2002; 45: 962-72, J Med Chem. 2002; 45: 5694-709, J Med Chem, 1998; 41: 183-90, J Med Chem. 1999; 42: 1625-38, J Med Chem. 2000; 43: 74655, J Med Chem. 2000; 43: 829-42, J Med Chem. 2003; 46: 4974-87, J Med Chem. 2001; 44: 340-9, Br J Pharmacol. 1998; 124: 1-3, Med Chem. 2002; 45: 208-18, Mol Pharmacol. 1996; 50: 1323-9, J Med Chem. 1998; 41: 1456-66, Br J Pharmacol. 2002; 135: 2004-10, J Med Chem. 1998; 41: 2201-6, J Med Chem. 2002; 45: 962-72, Eur J Pharmacol. 2001; 412: 213-21, Br J Pharmacol. 2002; 135: 1839-40, Br J Pharmacol. 1997; 121: 338-44, Int J Hematol. 2001; 74: 375-81, Arch Biochem Biophys. 2000; 381: 171-2, Cardiovasc Drug Rev. 2003; 21: 67-76, Curr Pharm Des. 2003; 9: 2303-16, Biochem Biophys Res Commun. 2000; 272: 327-31, Platelets. 2002; 13: 285-92, J Med Chem. 1999; 42: 5325-37, J Org Chem. 2002; 67: 8063-71, J Med Chem. 1999; 42: 5338-47, J Med Chem. 2002; 45: 5384-96, J Med Chem. 2001; 44: 3746-9, J Thromb Haemost. 2003; 1: 1144-9, J Auton Nerv Syst. 2000; 81: 171-7, Neurosci Lett. 2000; 284: 179-81, J Med Chem. 2000; 43: 746-55, and FEBS Lett. 1997; 403: 26-30, each of which is hereby incorporated by reference.

Thrombin inhibitors include bivalirudin, lepirudin, argatroban, melagatran, ximelagatran, antithrombin IIII, dermatan, mesoglycan, MB-015, BIBR 1048 (J Med Chem. 2002, 45:1757-66.), efegatran, TRI-50B, inogatran, V19, and PEG-r-hirudin. Additional thrombin inhibitors are disclosed in U.S. Pat. No. 6,680,312, U.S. Pat. No. 6,677,473, WO04002985A1, U.S. Pat. No. 6,673,912, U.S. Pat. No. 6,670,381, U.S. Pat. No. 6,664,255, U.S. Pat. No. 6,660,885, US20030225131A1, US20030220317A1, U.S. Pat. No. 6,653,316, US20030216301A1, EP0991624B1, EP1361212A1, EP1359913A1, WO03091284A1, EP1019047B1, U.S. Pat. No. 6,642,253, EP1355894A1, EP1355674A2, US20030191139A1, EP1110968B1, EP0739886B1, U.S. Pat. No. 6,624,180, EP0672658B1, U.S. Pat. No. 6,620,837, EP1341784A2, U.S. Pat. No. 6,617,317, US20030166579A1, U.S. Pat. No. 6,610,701, U.S. Pat. No. 6,610,692, US20030158218A1, U.S. Pat. No. 6,599,894, U.S. Pat. No. 6,599,881, U.S. Pat. No. 6,596,847, US20030134801A1, US20030118644A1, U.S. Pat. No. 6,579,867, WO03048155A1, U.S. Pat. No. 6,576,657, U.S. Pat. No. 6,569,851, EP1311480A1, US20030092679A1, U.S. Pat. No. 6,562,828, EP0858464B1, U.S. Pat. No. 6,559,141, EP1303491A1, EP0836615B1, U.S. Pat. No. 6,552,038, U.S. Pat. No. 6,552,030, EP0773955B1, U.S. Pat. No. 6,548,668, U.S. Pat. No. 6,544,978, EP0871464B1, U.S. Pat. No. 6,541,499, U.S. Pat. No. 6,541,467, U.S. Pat. No. 6,541,466, U.S. Pat. No. 6,539,944, WO03022873A1, EP0980367A4, EP0479489B1, U.S. Pat. No. 6,534,650, U.S. Pat. No. 6,534,639, U.S. Pat. No. 6,534,629, U.S. Pat. No. 6,534,536, U.S. Pat. No. 6,534,510, EP0934064B1, WO03018551A1, EP1287018A2, EP1021411B1, U.S. Pat. No. 6,528,505, U.S. Pat. No. 6,528,503, EP1283837A1, U.S. Pat. No. 6,521,625, WO03011222A2, EP 1189899B1, U.S. Pat. No. 6,518,310, U.S. Pat. No. 6,515,011, U.S. Pat. No. 6,514,978, EP0910573B1, WO03006462A1, U.S. Pat. No. 6,509,331, US20030013700A1, U.S. Pat. No. 6,506,754, US20030004308A1, EP 1267876A1, US20020193398A1, US20020193336A1, U.S. Pat. No. 6,495,574, EP0997460B1, U.S. Pat. No. 6,492,541, U.S. Pat. No. 6,492,402, U.S. Pat. No. 6,492,376, U.S. Pat. No. 6,492,368, EP0858262B1, U.S. Pat. No. 6,489,289, EP1259506A1, U.S. Pat. No. 6,486,129, US20020169318A1, US20020169189A1, US20020169113A1, EP0949921B1, EP0927035A4, EP0699074B1, U.S. Pat. No. 6,479,485, EP0816376B1, U.S. Pat. No. 6,476,016, U.S. Pat. No. 6,472,399, EP1079835A4, U.S. Pat. No. 6,469,029, WO02081465A1, EP0956049B1, U.S. Pat. No. 6,465,495, U.S. Pat. No. 6,465,459, U.S. Pat. No. 6,465,433, EP0772590B1, U.S. Pat. No. 6,462,050, U.S. Pat. No. 6,462,021, US20020142968A1, US20020142967A1, U.S. Pat. No. 6,458,952, U.S. Pat. No. 6,455,671, U.S. Pat. No. 6,455,532, EP0917528B1, U.S. Pat. No. 6,451,992, EP0635008B1, U.S. Pat. No. 6,444,817, U.S. Pat. No. 6,444,811, US20020119992A1, US20020119977A1, U.S. Pat. No. 6,441,008, WO02064559A2, WO02064211A1, WO02064140A1, U.S. Pat. No. 6,432,921, WO02060378A3, WO02060378A2, EP0479721B1, WO02059065A3, WO02059065A2, US20020103379A1, EP0993300A4, WO02057225A3, WO02057225A2, EP0973517A4, U.S. Pat. No. 6,417,161, WO02051824A1, WO02051445A3, WO02051445A2, EP1007548A4, WO0250056A1, U.S. Pat. No. 6,410,272, EP 1189618A4, U.S. Pat. No. 6,407,067, WO0246160A3, WO0246160A2, U.S. Pat. No. 6,391,901, U.S. Pat. No. 6,387,911, EP1200118A1, U.S. Pat. No. 6,380,221, U.S. Pat. No. 6,376,649, U.S. Pat. No. 6,376,523, U.S. Pat. No. 6,376,499, WO0230940A3, WO0230940A2, U.S. Pat. No. 6,372,751, U.S. Pat. No. 6,372,423, EP1195608A1, EP0863916B1, U.S. Pat. No. 6,369,209, US20020040043A1, EP1124822A4, EP1189899A1, EP1189618A1, EP0820287B1, U.S. Pat. No. 6,362,190, U.S. Pat. No. 6,362,166, WO0222584A1, US20020035115A1, EP1187616A1, U.S. Pat. No. 6,359,136, U.S. Pat. No. 6,358,922, U.S. Pat. No. 6,350,774, U.S. Pat. No. 6,350,764, U.S. Pat. No. 6,350,761, U.S. Pat. No. 6,350,746, U.S. Pat. No. 6,350,745, WO0214270A1, US20020022612A1, US20020019371A1, WO0209711A1, U.S. Pat. No. 6,344,466, U.S. Pat. No. 6,342,609, EP1017393A4, WO0204423A1, US20020006923A1, US20020004507A1, EP1169318A1, EP1117660A4, EP0934064A4, EP0918768B1, EP1165601, EP1082324A4, WO0192234A1, U.S. Pat. No. 6,326,492, U.S. Pat. No. 6,326,386, EP1017383A4, WO0187879A1, EP0900207B1, WO0185760A1, EP0799052B1, EP0652900B1, EP1150996A1, EP0864092B1, EP0815139131, WO0179262A1, WO0179261A1, US20010031757A1, EP0789585B1, U.S. Pat. No. 6,288,105, U.S. Pat. No. 6,284,756, U.S. Pat. No. 6,277,863, EP0841918B1, U.S. Pat. No. 6,271,227, U.S. Pat. No. 6,262,028, US20010007764A1, US20010006620A1, U.S. Pat. No. 6,239,132, U.S. Pat. No. 6,235,778, U.S. Pat. No. 6,204,384, U.S. Pat. No. 6,204,263, U.S. Pat. No. 6,201,006, U.S. Pat. No. 6,200,967, U.S. Pat. No. 6,174,855, EP0725797B1, WO0074682A1, U.S. Pat. No. 6,156,540, U.S. Pat. No. 6,147,078, WO0066152A1, WO0064470A1, WO0064449A1, WO0061609A3, WO0061609A2, U.S. Pat. No. 6,133,422, U.S. Pat. No. 6,133,297, U.S. Pat. No. 6,133,262, U.S. Pat. No. 6,127,337, U.S. Pat. No. 6,124,277, U.S. Pat. No. 6,121,435, U.S. Pat. No. 6,117,888, EP1023324A1, U.S. Pat. No. 6,096,877, EP1021411A1, U.S. Pat. No. 6,090,916, U.S. Pat. No. 6,090,787, U.S. Pat. No. 6,087,487, U.S. Pat. No. 6,087,375, U.S. Pat. No. 6,087,373, WO0039124A1, U.S. Pat. No. 6,083,971, EP1007548A1, U.S. Pat. No. 6,071,514, U.S. Pat. No. 6,069,232, U.S. Pat. No. 6,060,451, U.S. Pat. No. 6,057,314, WO0020394A1, U.S. Pat. No. 6,040,441, U.S. Pat. No. 6,037,356, U.S. Pat. No. 6,036,958, U.S. Pat. No. 6,034,104, U.S. Pat. No. 6,011,047, U.S. Pat. No. 6,011,038, EP0956049A2, U.S. Pat. No. 5,985,899, U.S. Pat. No. 5,985,833, U.S. Pat. No. 5,977,114, U.S. Pat. No. 5,965,692, U.S. Pat. No. 5,955,294, U.S. Pat. No. 5,945,275, U.S. Pat. No. 5,942,620, U.S. Pat. No. 5,939,392, U.S. Pat. No. 5,932,567, U.S. Pat. No. 5,914,319, EP0918768A1, EP0910629A1, EP0910583A1, WO9919356A1, U.S. Pat. No. 5,891,909, U.S. Pat. No. 5,885,967, U.S. Pat. No. 5,877,156, U.S. Pat. No. 5,876,971, U.S. Pat. No. 5,874,407, U.S. Pat. No. 5,872,098, U.S. Pat. No. 5,866,543, U.S. Pat. No. 5,866,542, EP0739886A3, U.S. Pat. No. 5,864,009, U.S. Pat. No. 5,863,929, U.S. Pat. No. 5,863,894, U.S. Pat. No. 5,856,307, EP0884325A1, U.S. Pat. No. 5,840,733, EP0841918A4, EP0863916A1, EP0670310B1, U.S. Pat. No. 5,798,377, EP0858464A1, U.S. Pat. No. 5,795,896, U.S. Pat. No. 5,783,563, EP0841918A1, U.S. Pat. No. 5,747,535, U.S. Pat. No. 5,747,291, WO9816252A1, EP0837071A1, U.S. Pat. No. 5,741,819, U.S. Pat. No. 5,741,799, U.S. Pat. No. 5,741,792, U.S. Pat. No. 5,736,364, EP0832879A1, WO9812319A1, U.S. Pat. No. 5,728,549, U.S. Pat. No. 5,726,159, U.S. Pat. No. 5,723,576, U.S. Pat. No. 5,723,444, U.S. Pat. No. 5,714,485, U.S. Pat. No. 5,710,130, U.S. Pat. No. 5,707,966, WO9800443A1, WO9800171A2, EP0815139A1, U.S. Pat. No. 5,705,487, WO9749801A1, WO9749673A1, EP0625908B1, U.S. Pat. No. 5,698,104, WO9745424A1, EP0809651A1, U.S. Pat. No. 5,691,356, EP0536177B1, U.S. Pat. No. 5,686,564, U.S. Pat. No. 5,681,925, U.S. Pat. No. 5,670,479, U.S. Pat. No. 5,661,001, EP0789585A1, U.S. Pat. No. 5,656,600, WO9722589A1, WO9717363A1, U.S. Pat. No. 5,629,324, U.S. Pat. No. 5,627,038, WO9712904A1, U.S. Pat. No. 5,612,378, U.S. Pat. No. 5,612,369, U.S. Pat. No. 5,610,308, WO9705160A1, U.S. Pat. No. 5,602,253, U.S. Pat. No. 5,602,101, U.S. Pat. No. 5,599,793, U.S. Pat. No. 5,597,804, WO9640118A1, WO9639132A1, U.S. Pat. No. 5,583,146, U.S. Pat. No. 5,583,113, U.S. Pat. No. 5,578,574, EP0739886A2, EP0686642A3, U.S. Pat. No. 5,561,146, WO9629347A1, U.S. Pat. No. 5,559,150, U.S. Pat. No. 5,547,850, WO9624609A1, EP0725797A1, EP0601459A3, U.S. Pat. No. 5,510,369, EP0626070B1, U.S. Pat. No. 5,488,037, U.S. Pat. No. 5,484,772, EP0686642A2, EP0683674A1, EP0670310A1, U.S. Pat. No. 5,443,827, U.S. Pat. No. 5,439,888, U.S. Pat. No. 5,436,229, EP0663009A1, U.S. Pat. No. 5,433,940, U.S. Pat. No. 5,430,023, U.S. Pat. No. 5,425,936, U.S. Pat. No. 5,422,249, U.S. Pat. No. 5,416,093, WO9511921A1, EP0648780A1, WO9504823A1, U.S. Pat. No. 5,371,091, EP0623596A1, EP0623595A1, U.S. Pat. No. 5,332,822, EP0603112A1, EP0601459A2, EP0479489A3, U.S. Pat. No. 5,252,566, U.S. Pat. No. 5,240,913, EP0536177A1, EP0479489A2, WO9119734A2, U.S. Pat. No. 5,030,631, and EP0168342B1, each of which is hereby incorporated by reference.

CD40 antagonists include soluble CD40 ligand and CD40 antibody. Additional CD40 antagonists are disclosed in WO0228481A3, WO0228481A2, WO0228480A3, WO0228480A2, WO03028809A1, U.S. Pat. No. 5,683,693, WO0134649A3, WO0134649A2, WO0124823A1, EP1274455A1, EP1221973A1, U.S. Pat. No. 5,942,229, EP0797446A1, WO9616665A1, U.S. Pat. No. 6,682,739, WO03086463A1, U.S. Pat. No. 6,635,743, US20030165499A1, WO03062262A2, WO03045978A3, WO03045978A2, WO03029296A1, WO02092761A3, WO02092761A2, WO0211763A1, US2002000976A1, U.S. Pat. No. 6,265,556, WO0134194A1, U.S. Pat. No. 6,172,187, US20040006208A1, US20030180292A1, EP1322383A2, US20030113341A1, U.S. Pat. No. 6,534,061, US20020150559A1, U.S. Pat. No. 6,458,839, WO0228905A3, WO0228905A2, WO0228904A3, WO0228904A2, US20020031512A1, US20010018041A1, EP0721346A1, US20030180292A1, and US20020058029A1, each of which is hereby incorporated by reference.

Examples of leukotriene inhibitors include monelukast, zafirlukast, and zileuton, Other leukotriene inhibitors are described in EP0378765B1, EP0378765A1, US20010025040A1, WO0160407A3, WO01060407A2, EP0748312B1, EP0748312A1, U.S. Pat. No. 5,508,408, WO9523789A1, EP0342664B1, EP0342664A3, EP0342664A2, WO8904305A1, WO8904304A1 or WO08904303A1, each of which is hereby incorporated by reference.

Coadministration. The platelet activation or aggregation inhibitors described herein may be administered as a monotherapy or in combination. In addition, platelet activation or aggregation inhibitors may be administered with other compounds, such as those that lower cholesterol, e.g., statins (such as, atorvastatin, fluvastatin, lovastatin, pravastatin, cerivastatin, rosuvastatin, and simvastatin), nicotinic acid drugs (such as, Advicor, Niacin, and Niaspin), drugs that sequester bile acid (such as, colestipol, cholestyramine, and colesevelam), fibrates (such as, clofibrate, gemfibrozil, and fenofiribrate), ETC 588, ETC 216, ETC 642, and pirozadil. Other drugs are described in US20040018210A1, U.S. Pat. No. 6,682,913, US20040010047A1, U.S. Pat. No. 6,669,955, WO03103640A1, WO03103633A1, WO03103632A1, EP1370210A2, U.S. Pat. No. 6,664,281, US20030225145A1, EP1362855A1, EP1357927A1, EP1354879A1, U.S. Pat. No. 6,610,320, US20030153579A1, U.S. Pat. No. 6,605,615, US20030144341A1, U.S. Pat. No. 6,589,969, U.S. Pat. No. 6,576,256, US20030105028A1, EP 1222302A4, WO03042194A1, EP 1206939A4, US20030068366A1, WO03027073A1, US20030060477A1, WO03020243A1, US20030049314A1, EP1285650A1, WO03013559A1, U.S. Pat. No. 6,512,008, U.S. Pat. No. 6,506,757, EP 1272219A1, WO02102780A1, US20020173535A1, US20020155091A1, WO02081528A1, US20020137690A1, WO02067895A2, WO02066464A1, WO02060465A1, EP1228765A1, EP1222302A1, WO02051820A1, WO0250090A1, EP1122255A4, EP0877750B1, EP1206939A1, U.S. Pat. No. 6,388,131, U.S. Pat. No. 6,372,462, U.S. Pat. No. 6,365,186, U.S. Pat. No. 6,362,236, US20020013496A1, WO0197751A3, WO0197751A3, WO0197751A3, US20010056096A1, WO0185155A1, U.S. Pat. No. 6,316,041, EP1150679A1, WO0178529A3, WO0178529A2, WO0174394A1, U.S. Pat. No. 6,297,268, U.S. Pat. No. 6,294,190, WO0164646A3, WO0164646A, US20010016197A1, EP1122255A1, EP1108713A1, WO0127305A1, EP0815857A4, U.S. Pat. No. 6,204,032, WO115674A2, U.S. Pat. No. 6,190,720, WO0105412A1, U.S. Pat. No. 6,174,560, EP1067109A1, WO0069445A1, EP1047421A2, U.S. Pat. No. 6,133,001, WO0060107A1, EP1005453A1, EP0636367B1, U.S. Pat. No. 6,043,389, U.S. Pat. No. 6,037,379, U.S. Pat. No. 5,958,913, U.S. Pat. No. 5,939,455, WO09926583A2, EP0917587A1, EP0900077A1, WO9910320A1, EP0807435A3, EP0812840A4, EP0637447B1, EP0877750A1, U.S. Pat. No. 5,830,902, U.S. Pat. No. 5,756,470, EP0713706A3, EP0815857A1, EP0812840A1, U.S. Pat. No. 5,698,527, EP0807435A2, WO9732574A1, U.S. Pat. No. 5,662,934, U.S. Pat. No. 5,618,707, EP0586518B1, EP0492474B1, U.S. Pat. No. 5,607,965, WO09626938A1, U.S. Pat. No. 5,523,460, EP0713706A2, U.S. Pat. No. 5,506,262, U.S. Pat. No. 5,475,029, U.S. Pat. No. 5,466,815, EP0678020A1, U.S. Pat. No. 5,447,726, U.S. Pat. No. 5,447,717, U.S. Pat. No. 5,441,731, EP0637447A3, EP0537300B1, U.S. Pat. No. 5,409,820, WO9504025A1, EP0637447A2, EP0636367A1, U.S. Pat. No. 5,369,125, U.S. Pat. No. 5,364,644, EP0617000A3, EP0617000A2, U.S. Pat. No. 5,332,857, WO9415911A1, WO9415592A1, U.S. Pat. No. 5,326,783, WO9409774A1, WO9408940A1, U.S. Pat. No. 5,302,604, EP586518A1, U.S. Pat. No. 5,294,627, WO9404144A1, U.S. Pat. No. 5,284,971, WO9402134A1, U.S. Pat. No. 5,283,256, WO9401104A1, U.S. Pat. No. 5,278,320, U.S. Pat. No. 5,270,332, U.S. Pat. No. 5,264,593, U.S. Pat. No. 5,260,215, U.S. Pat. No. 5,258,401, U.S. Pat. No. 5,256,689, EP334673B1, U.S. Pat. No. 5,254,727, U.S. Pat. No. 5,252,471, WO9317557A1, U.S. Pat. No. 5,244,887, EP018181B1, EP0526936A3, U.S. Pat. No. 5,208,331, EP0537300A1, U.S. Pat. No. 5,204,336, EP0530242A1, EP0526936A2, U.S. Pat. No. 5,182,298, U.S. Pat. No. 5,178,854, EP0505135A3, WO9221336A1, U.S. Pat. No. 5,169,976, EP0512865A3, WO9220336A2, U.S. Pat. No. 5,166,171, EP0512865A2, U.S. Pat. No. 5,158,949, U.S. Pat. No. 5,151,545, EP0505135A2, U.S. Pat. No. 5,135,945, U.S. Pat. No. 5,132,320, EP0492474A1, U.S. Pat. No. 5,118,697, EP0462021A3, U.S. Pat. No. 5,102,917, EP0475706A1, U.S. Pat. No. 5,096,923, EP0402062A3, EP0402062A3, EP0403271A3, EP0403199A3, EP0403198A3, WO9119704A2, EP0462021A2, U.S. Pat. No. 5,072,002, WO9118027A2, U.S. Pat. No. 5,037,842, EP0334673A3, EP0403271A2, EP0403199A2, EP0403198A2, EP0402062A2, EP0318181A3, EP0296622A3, EP0334673A, WO8904821A1, EP0318181A2, WO8903212A1, EP0296622A2, U.S. Pat. No. 4,759,923, EP0026851B1, EP0026851A3, EP0026851A2, U.S. Pat. No. 4,117,159, U.S. Pat. No. 4,069,338, U.S. Pat. No. 4,044,043, U.S. Pat. No. 4,009,206, U.S. Pat. No. 3,821,378, U.S. Pat. No. 3,682,963, U.S. Pat. No. 3,674,842, U.S. Pat. No. 6,689,590, U.S. Pat. No. 6,686,481, U.S. Pat. No. 6,686,185, US20040018248A1, EP1383447A1, WO04004774A2, U.S. Pat. No. 6,673,831, US20030229015A1, WO034923A1, EP1361867A1, U.S. Pat. No. 6,649,775, WO03092729A1, US2003211151A1, EP1359940A2, EP1358343A2, EP1353727A2, US20030195145A1, US20030186994A1, WO03075933A1, US20030175237A1, US20030171407A1, WP1340752A1, EP1339429A2, US20030162807A1, US20030158426A1, WO03061702A1, EP1326591A2, EP1322306A2, EP1291017A3, U.S. Pat. No. 6,586,627, US20030114685A1, US20030114515A1, WO03048149A1, WO03048148A3, WO03048148A2, US20030108657A1, US20030108657A1, US20030105154A1, US20030104004A1, WP1316552A1, US20030100493A1, U.S. Pat. No. 6,569,835, U.S. Pat. No. 6,569,461, US20030092745A1, U.S. Pat. No. 6,534,088, EP129017A2, WO03007846A1, U.S. Pat. No. 6,511,800, WO03006656A3, WO03006656A2, WO03004456A1, WO03004455A3, WO03004455A2, WO03004450A1, WO02100394A1, EP1265841A2, EP1265604A1, US20020187531A1, US20020177610A1, WO02087472A1, EP1253922A2, US20020161032A1, US20020159974A1, US20020159973A1, WO02083124A1, US20020156122A1, U.S. Pat. No. 6,465,454, U.S. Pat. No. 6,455,574, EP 1071403A4, WO02067901A1, WO02064809A3, WO02064809A2, US20020115712A1, US20020103252A1, US20020090622A1, US20020086889A1, US20020077317A1, WO0243807A3, WO0243659A3, WO0243659A2, U.S. Pat. No. 6,391,583, U.S. Pat. No. 6,388,100, U.S. Pat. No. 6,387,258, WO0232411A3, WO023241A2WO0232408A3, US20020049237A1, U.S. Pat. No. 6,367,479, WO0224194A3, WO0224194A2, US20020034546A1, US20020025981A1, US20020025952A1, US20020019391A1, WO0209697A1, WO0206266A1 WO0203985A1, WO0203977A3, WO0203977A2, US20020002288A1, EP1150665A2, US20010036936A1, U.S. Pat. No. 6,294,680, WO0166538A1, EP1127573A1, EP1123717A2, U.S. Pat. No. 6,268,186, WO0152829A3, WO0152829A2, US20010006697A1, US20010005714A1, WO0144144A3, WO0144144A2, WO0137831A1, U.S. Pat. No. 6,235,311, WO0128499A3, WO0128499A2, WO0122976A2, EP1071403A1, U.S. Pat. No. 6,180,680, U.S. Pat. No. 6,180,660, U.S. Pat. No. 6,177,121, WO0064443A1, EP1045691A1, U.S. Pat. No. 6,133,312, EP1037623A1, WP1036563A1, WO0053173A1, WO0048626A2, WO0047196A3, U.S. Pat. No. 6,079,416, EP1003507A1, EP0983066A1, EP0971913A1, WO9947123A1, U.S. Pat. No. 5,939,587, U.S. Pat. No. 5,932,749, WO9930706A1, WO9,930,704A1, WO9911263A1, WO9910499A1, WO9901126A1, EP0889906A1, WO9832751A1, WO9736927A1, and U.S. Pat. No. 3,621,043, each of which is hereby incorporated by reference.

Additional agents that may be co-administered include compounds acting to modify eicosanoid activity may also be administered with the therapeutic agents described above. Compounds acting to modify eicosanoid activity may include COX inhibitors, PGE1 agonists, PG synthase inhibitors, TX synthase inhibitors, and TXA2 antagonists. Examples of these compounds include iloprost, epoprost, picotamide, indometacin farnesil, trifusal, pamicogrel, alprostadil, limaprost, carbasalate, indobufen, ozagrel, etersalate, cloricomene, beraprost, Z-335, terbogrel, and ramatroban.

Further agents that can be co-administered with other therapeutic compounds include 5HT2a antagonists (e.g., sarpogrelate), nonsteroidal anti-inflammatory drugs (e.g., ditazole), adrenergic inhibitors (e.g., chlorthalidone, clonidine, doxazosin, guanzbenz, guanadrel, guanethidine, guanfacine, methyldopa, phenoxybenzamine, polythiazide/prazosin, prazosin, reserpine, and terazosin), angiotensin converting enzyme inhibitors (e.g., benazepril, captopril, cilazapril, enalapril, fosinopril, lisinopril, moexipril, perindopril, quinapril, ramipril, spirapril, temocapril, and trandolapril), angiotensin II receptor antagonists (e.g., candesartan, eprosartan, irbesartan, losartan, and valsartan), fibrilyinic agents (e.g., reteplase, streptokinea, urokinase, and tenecteplase), beta blockers (e.g., acebutolol, betaxolol, bisopropol, carvediol, dilevalol, esmolol, labetalol, levobunolol, metipranolol, metoprolol, nadolol, penbutolol, pindolol, propranolol, and timolol), calcium channel blockers (e.g., amlodipine, amlodipine/benzapril, bepridil, diltiazem, felodipine, imidapril, isradipine, isosorbide, manidipine, nicardipine, nifedipine, nilvadipine, nimodipine, nisoldipine, and verapamil), diuretic agents (e.g., acetazolamide, amiloride, bendroflumethiazide, benzthiazide, bumetanide, chlorothiazide, chlorthalidone, cyclothiazide, ethacrynic acid, furosemide, hydrochlorothiazide, mannitol, methylchlothiazide, metolazone, milrinone, polythiazide, potassium chloride, spironolactone, torsemide, triamterene, and trichlormethiazide), steroids and steroidal glycosides (e.g., convallaria, crataegus, digitalis, ouabain, and strophantin), and vasodilators (e.g., alprostadil, amyl nitrate, cilostazol, cyclandelate, diazoxide, ethaverine, flosequinan, hydrazaline, isoxsuprine, minoxidil, nitroglycerin, papaverine, pentoxifyline, sodium nitroprusside, and tolazoline).

Two or more agents may be administered concomitantly in the same dose or in separate doses. Agents in combination may also be administered at different times as appropriate. In one embodiment, a glycoprotein IIb/IIIa receptor antagonist, e.g., xemilofiban, is co-administered with aspirin and a heparin, e.g., a low molecular weight heparin.

### Administration

Therapeutic compounds may be administered in pharmaceutically acceptable carriers, such as sterile water or isotonic saline. Unless otherwise specified, the compounds of the invention can be administered by any standard means, including, without limitation, oral, sublingual, transdermal, intravenous, parenteral, subcutaneous, intramuscular, intraperitoneal, intracoronary infusion, and administration into the cerebrospinal fluid, Exemplary forms of transdermal delivery include a patch, sonophoresis, a microneedle array, and iontophoresis (e.g., as disclosed in U.S. Pat. No. 5,961,482). Administrations may be accomplished using standard means, such as auto-injection devices, constant infusion pumps, and minipumps. The compounds described herein may also be impregnated or coated on a medical device, such as a stent, as disclosed in U.S. Pat. No. 5,609,629, hereby incorporated by reference.

Dosages and timing of administration can be determined using routine methods for such determination. Therapeutic compounds may be administered, e.g., once, twice, three times, four times, or more a day. The compounds may also be delivered continuously, e.g., through continuous infusion iontophoresis, subcutaneous, or time-release formulations, over a period of time. For patients at high risk, e.g., diabetics, the treatment may be for 30 days or more. Typically, a 30-day treatment of high risk patients will also include a direct thrombin inhibitor or a Factor Xa inhibitor. The exact times of administration after the procedure may depend on the patient and the circumstances, e.g., the type of medical or surgical procedure being employed or the medical condition requiring treatment, e.g., acute myocardial infarction. The administration of antagonist administered after a procedure and during or subsequent to an adverse medical condition is designed to minimize prothrombotic events. In one embodiment, a bolus is administered as soon as practicable after the insertion of a diagnostic catheter. The amount of antagonist administered is, e.g., 0.5 mg/dose, 1 mg/dose, 2.5 mg/dose, 5 mg/dose, 10 mg/dose, 20 mg/dose, 40 mg/dose, or even 80 mg/dose. Other dosages may be determined by one skilled in the art. The dosage regimen may be designed to prevent “troughs” or reduced periods of platelet inhibition that may be prothrombotic. In addition, it may be desirable to dose the patients in order to provide for rapid reversal of anti-thrombotic activity. Treatment may, for example, inhibit at least 60%, 70%, 80%, 90%, or 95% of platelet aggregation in a patient.

In one embodiment, a first platelet activation or aggregation inhibitor is administered intravenously, subcutaneously, or transdermally before a medical or surgical procedure, e.g., at least 30 min, or 1, 2, 6, 12, 24, or 48 hours, or within 10, 20, or 30 min, or 1, 2, 6, 12, or 48 hours of diagnosing an adverse medical condition, such as acute myocardial infarction. The initial intravenous, subcutaneous or transdermal dose may also be employed during transportation to or between medical facilities. For example, an intravenous, subcutaneous, or transdermal administration can occur during an initial ambulance ride to a hospital, or during the transportation of a patient from a rural hospital to a cardiac care facility. The initial dosage is typically administered as a bolus, i.e., a loading dose. The initial treatment preferably inhibits at least 80% of platelet aggregation and more preferably at least 90% (Jennings et al. J. Interv. Cardiol. 2002; 15: 45-60).

A loading dose allows the maximal plasma concentration to be achieved in the shortest time frame potentially conferring the greatest degree of protection to the patient by maximally inhibiting platelet activation or aggregation. Without a loading dose, steady state plasma concentrations may not be achieved until 4-5 plasma half-lives or about 24-30 hours. Preferably, intravenous, subcutaneous, and transdermal administration provide the same physiological level of the therapeutic agent.

Once the medical or surgical procedure is completed or the adverse medical condition has been initially treated, treatment continues with oral administration of a platelet activation or aggregation inhibitor. Oral treatment provides a more cost effective and less invasive manner of managing the long term treatment of a patient, compared to IV administration. Other advantages are described herein.

In one example, a first platelet activation or aggregation inhibitor is administered as a loading dose via IV bolus, A second platelet activation or aggregation inhibitor is then administered orally for at least 2 days, e.g., at least 7, 14, or 30 days. The bolus may deliver 0.3 to 60 mg, e.g., 1 to 10 mg or 3 to 6 mg, of the first platelet activation or aggregation inhibitor. The dosage of the second platelet activation or aggregation inhibitor may be selected to maintain the same level of inhibition of platelet aggregation achieved by the bolus. Alternatively, the dosing of the second inhibitor may be selected to increase or decrease the level of inhibition of platelet aggregation.

In another example, the first platelet activation or aggregation inhibitor is administered as a continuous intravenous, subcutaneous, or transdermal infusion, e.g., for at least 6, 12, 18, 24, or 48 hours. The continuous infusion may deliver 0.01 to 1.0 mg/kg/min, e.g., about 0.05, 0.06, 0.07, 0.08, 0.09, 0.1, 0.110, 0.120, 0.125, 0.130, 0.135, 0.140, 0.145, 0.150, 0.160, 0.170, 0.180, 0.190, or 0.2 mg/kg/min, of the first platelet activation or aggregation inhibitor. Preferably, the total amount of the first platelet activation or aggregation inhibitor administered is 0.3 to 60 mg, e.g., 1 to 10 mg. After the intravenous, subcutaneous, or transdermal infusion, a second platelet activation or aggregation inhibitor is then administered orally for at least 1, 2, 7, 14, or 30 days. The dosage of the second platelet activation or aggregation inhibitor may be selected to maintain the same level of inhibition of platelet aggregation achieved by the continuous infusion. Alternatively, the dosing of the second inhibitor may be selected to increase or decrease the level of inhibition of platelet aggregation.

In another embodiment, therapy includes administration of a platelet activation or aggregation inhibitor subcutaneously, a Factor Xa inhibitor, a heparin, and a thrombin inhibitor to a subject. The agents may be administered in any order. Dosing typically occurs twice a day for at least a 30 day period. Other dosing regimes can be determined by one skilled in the art.

The dosing may also depend on the body mass index of the patient. For example for a subject having a body mass index (BMI) of greater than 25 (e.g., greater than 30), 4 to 10 mg of the first platelet activation or aggregation inhibitor may be administered, and for a subject having a BMI of less than 30 (e.g., less than 25), 1 to 3 mg of the first platelet activation or aggregation inhibitor may be administered. The dosage of the second platelet activation or aggregation inhibitor may also similarly depend on the BMI of the subject. For example, for subjects having a BMI less than 30 kg/m² (e.g., less than 25 kg/m²) a 30-40 mg loading dose is followed by oral administration of 20 mg TID or QID of a therapeutic agent. For subjects having a BMI greater than 25 (e.g., greater than 30) kg/m², a 40-50 mg loading dose is followed by oral administrations of 20 mg TID or QID of a therapeutic agent.

### Medical or Surgical Procedures

Medical or surgical procedures that may cause unwanted platelet aggregation include, for example, cardiovascular interventional procedures. These procedures include, without limitation, percutaneous transluminal coronary angioplasty (PTCA), percutaneous coronary intervention (PCI), coronary artery stent procedure, cardiac bypass surgery (CABG), peripheral transluminal angioplasty (PTA), peripheral vascular stent implantation, and an angioplasty procedure. An angioplasty procedure may be, for example, an atherectomy, balloon angioplasty, laser angioplasty, intracranial angioplasty, or angioplasty of peripheral arteries. The medical or surgical procedure may include the insertion of a coronary catheter, e.g., a diagnostic catheter, into a subject or the implantation of a stent that may or may not be a drug-eluting stent.

The following non-limiting examples illustrate various embodiments of the invention.

### Example 1

A patient is admitted to a hospital to undergo a cardiac interventional, e.g., percutaneous transluminal coronary angioplasty, a percutaneous coronary intervention, a coronary artery stent procedure, coronary artery bypass surgery, peripheral transluminal angioplasty, peripheral vascular stent implantation, or an angioplasty procedure. Thirty minutes prior to the initiation of the procedure, the patient is administered an intravenous bolus loading dose of 1 to 10 mg of xemilofiban, e.g., 3 to 6 mg of xemilofiban, with the actual amount administered being adjusted according to the body mass index of the individual patient and according to the amount of platelet inhibition desired, e.g., greater than 80, 85, or 90 percent inhibition of platelet aggregation. Following completion of the procedure, the patient is administered oral xemilofiban 10 to 40 mg four times daily (QID) for two days, with the dosage being adjusted to maintain plasma concentrations of 0.3 to 3000 ng/ml, e.g., 3 to 300 ng/ml or 30 to 90 ng/ml, and platelet inhibition of at least 80, 85, or 90 percent inhibition of platelet aggregation.

### Example 2

A patient is admitted to a hospital to undergo a cardiac interventional procedure consisting of percutaneous transluminal coronary angioplasty, a percutaneous coronary intervention, a coronary artery stent procedure, coronary artery bypass surgery, peripheral transluminal angioplasty, peripheral vascular stent implantation, or an angioplasty procedure. Thirty minutes prior to the initiation of the procedure, the patient is administered a subcutaneous bolus loading dose of 0.1 to 50 mg of xemilofiban, e.g., 1 to 10 mg of xemilofiban or 3 to 6 mg of xemilofiban, with the actual amount administered being adjusted according to the body mass index of the individual patient and according to the amount of platelet inhibition desired, e.g., greater than 80, 85, or 90 percent inhibition of platelet aggregation. Following completion of the procedure, the patient is administered oral xemilofiban 10 to 40 mg four times daily (QID) for two days, with the dosage being adjusted to maintain plasma concentrations of 0.3 to 3000 ng/ml, e.g., 3 to 300 ng/ml or 30 to 90 ng/ml, and platelet inhibition of at least 80, 85, or 90 percent inhibition of platelet aggregation.

### Example 3

A patient is admitted to a hospital to undergo a cardiac interventional procedure consisting of percutaneous transluminal coronary angioplasty, a percutaneous coronary intervention, a coronary artery stent procedure, coronary artery bypass surgery, peripheral transluminal angioplasty, peripheral vascular stent implantation, or an angioplasty procedure. Thirty minutes prior to the initiation of the procedure, the patient is administered a transdermal dose of 0.1 to 50 mg of xemilofiban, e.g., 1 to 10 mg or 3 to 6 mg of xemilofiban, with the actual amount administered being adjusted according to the body mass index of the individual patient and according to the amount of platelet inhibition desired, e.g., greater than 80, 85, or 90 percent inhibition of platelet aggregation. Following completion of the procedure, the patient is administered oral xemilofiban 10 to 40 mg four times daily (QID) for two days with the dosage being adjusted to maintain plasma concentrations of 0.3 to 3000 ng/ml, e.g., 3 to 300 ng/ml or 30 to 90 ng/ml, and platelet inhibition of at least 80, 85, or 90 percent inhibition of platelet aggregation.

### Other Embodiments

Modifications and variations of the described methods of the invention will be apparent to those skilled in the art without departing from the scope and spirit of the invention. Although the invention has been described in connection with specific desirable embodiments, it should be understood that the invention as claimed should not be unduly limited to such specific embodiments. Indeed, various modifications of the described modes for carrying out the invention, which are obvious to those skilled in the art, are intended to be within the scope of the invention.

All publications, patents, and patent applications mentioned in this specification are herein incorporated by reference to the same extent as if each individual publication, patent, or patent application was specifically and individually to be incorporated by reference.

Other embodiments are within the claims.

What is claimed is:

1. A method of inhibiting platelet aggregation in a subject, said method comprising the steps of:
(a) intravenously, subcutaneously, or transdermally administering a first platelet activation or aggregation inhibitor to said subject; and
(b) orally administering a second platelet activation or aggregation inhibitor to said subject, thereby inhibiting platelet aggregation in said subject, wherein said first and second platelet activation or aggregation inhibitors are the same or different, provided that, when said first platelet activation or aggregation inhibitor is heparin, said second platelet activation or aggregation inhibitor is not aspirin, and provided that when said first platelet activation or aggregation inhibitor is RPR 109891, said second platelet activation or aggregation inhibitor is not RPR 109891.

2. The method of claim 1, wherein said first platelet activation or aggregation inhibitor is a GP IIb/IIIa antagonist.

3. The method of claim 2, wherein said GP IIb/IIIa antagonist is tirofiban, abciximab, eptifibatide, TRM-147, SM-20302, L-378167, rClf A, ME-3230, SR-121787, UR-12947, L-734217, DMP-757, EMD-96717, SDZ-GPI-562, RG-13965, SB-207448, SC-56929, RWJ-50042, UR 4005, L-703014, SKF-106760, CRL-42796, HMR-1794, CGH-400, Ro-43-5054, Barbourin, Bitistatin, SC-49992, TP-9201, MA-16N7C2, roxifiban (DMP-754), lamifiban, xemilofiban, lotrafiban, sibrafiban, DU-728, DMP-728, MK-852, SC-52012A, echistatin, TAK-029, ME-3277, T-250, MS-180, TA-993, elarofiban (RWJ-53308), cromafiban (CT-50352), YM-337, lefradafiban (BIBU-104), fradafiban (BIBU-52), ZD-2486, RPR-109891, gantofiban, GR-144053F, or a pharmaceutically acceptable salt thereof.

4. The method of claim 2, wherein said GP IIb/IIIa antagonist is xemilofiban.

5. The method of claim 1, wherein said first platelet activation or aggregation inhibitor is selected from the group consisting of a heparin, tissue plasminogen activator, a Factor Xa inhibitor, a purinergic-receptor antagonist, a thrombin inhibitor, a phosphodiesterase inhibitor, a cyclooxygenase inhibitor, a CD40 antagonist, and a leukotriene inhibitor.

6. The method of claim 1, wherein said second platelet activation or aggregation inhibitor is a GP IIb/IIIa antagonist.

7. The method of claim 6, wherein said GP IIb/IIIa antagonist is tirofiban, abciximab, eptifibatide, TRM-147, SM-20302, L-378167, rClf A, ME-3230, SR-121787, UR-12947, L-734217, DMP-757, EMD-96717, SDZ-GPI-562, RG-13965, SB-207448, SC-56929, RWJ-50042, UR 4005, L-703014, SKF-106760, CRL-42796, HMR-1794, CGH-400, Ro-43-5054, Barbourin, Bitistatin, SC-49992, TP-9201, MA-16N7C2, roxifiban (DMP-754), lamifiban, xemilofiban, lotrafiban, sibrafiban, DU-728, DMP-728, MK-852, SC-52012A, echistatin, TAK-029, ME-3277, T-250, MS-180, TA-993, elarofiban (RWJ-53308), cromafiban (CT-50352), YM-337, lefradafiban (BIBU-104), fradafiban (BIBU-52), ZD-2486, RPR-109891, gantofiban, GR-144053F or a pharmaceutically acceptable salt thereof.

8. The method of claim 7, wherein said GP IIb/IIIa antagonist is xemilofiban.

9. The method of claim 1, wherein said second platelet activation or aggregation inhibitor is selected from the group consisting of a heparin, tissue plasminogen activator, a Factor Xa inhibitor, a purinergic-receptor antagonist, a thrombin inhibitor, a phosphodiesterase inhibitor, a cyclooxygenase inhibitor, a CD40 antagonists, and a leukotriene inhibitor.

10. The method of claim 1, wherein said first platelet activation or aggregation inhibitor is a first GP IIb/IIIa antagonist, and said second platelet activation or aggregation inhibitors is a second GP IIb/IIIa antagonist.

11. The method of claim 1, wherein said first platelet activation or aggregation inhibitor is administered as a loading dose.

12. The method of claim 11, wherein said first platelet activation or aggregation inhibitor is xemilofiban.

13. The method of claim 11, wherein said second GP IIb/IIIa antagonist is administered for at least 2 days, is administered for at least 7 days, is administered for at least 14 days, or is administered for at least 30 days.

14. The method of claim 11, wherein 0.3 to 60 mg of said first platelet activation or aggregation inhibitor is administered or wherein 1 to 10 mg of said first platelet activation or aggregation inhibitor is administered.

15. The method of claim 11, wherein said second platelet activation or aggregation inhibitor is xemilofiban.

16. The method of claim 1, wherein said first platelet activation or aggregation inhibitor is administered as a continuous infusion.

17. The method of claim 16, wherein said continuous infusion is administered intravenously, is administered subcutaneously, is administered transdermally, is administered by a patch, sonophoresis, a microneedle array, or iontophoresis.

18. The method of claim 16, wherein said first platelet activation or aggregation inhibitor is administered for at least 6 hours, is administered for at least 12 hours, is administered for at least 18 hours, is administered for at least 24 hours, or is administered for at least 48 hours.

19. A method of inhibiting platelet aggregation in a subject, said method comprising the steps of:
(a) subcutaneously administering a platelet aggregation inhibitor to said subject;
(b) administering a Factor Xa inhibitor to said subject;
(c) administering a heparin to said subject; and
(d) administering a thrombin inhibitor to said subject, thereby inhibiting platelet aggregation in said subject.

20. A kit comprising:
(a) a first platelet activation or aggregation inhibitor formulated for intravenous, transdermal, or subcutaneous administration; and
(b) a second platelet activation of aggregation inhibitor formulated for oral administration,
wherein said first and second platelet activation or aggregation inhibitors are the same or different.
